(19) United States
(12) Patent Application Publication
(10) Pub. No.: US 2022/0167926 A1
(43) Pub. Date: Jun. 2, 2022

(54) PATIENT POSITIONING APPARATUS FOR A MEDICAL IMAGING DEVICE

(71) Applicant: Siemens Healthcare GmbH, Erlangen (DE)

(72) Inventor: Arne Hengerer, Moehrendorf (DE)

(73) Assignee: Siemens Healthcare GmbH, Erlangen (DE)

(21) Appl. No.: 17/540,347

(22) Filed: Dec. 2, 2021

(30) Foreign Application Priority Data
Dec. 2, 2020 (DE) 102020215184.9

(51) Int. Cl. A61B 5/00

(52) U.S. Cl. CPC A61B 5/704

(57) ABSTRACT

A patient positioning apparatus may include a compressor, a support surface having an upper side with at least one upper opening pore and a lower side with at least two lower opening pores, and a valve having at least one first setting connecting the compressor to at least one lower opening pore of the at least two lower opening pores by a first line and a second setting connecting the compressor to the at least one upper opening pore by a second line. The compressor can create an overpressure in the first line and/or the second line, and/or create a vacuum in the first line and/or the second line.

### CROSS REFERENCE TO RELATED APPLICATIONS

This patent application claims priority to German Patent Application No. 10 2020 215 184.9, filed Dec. 2, 2020, which is incorporated herein by reference in its entirety.

### BACKGROUND

### Field

The disclosure relates to a patient positioning apparatus and to a medical imaging device.

### Related Art

Depending on a medical procedure required it is necessary to move a patient between different medical devices, for example between two different imaging modalities, in a lying position. It can be necessary within the framework of a radiation therapy to record specific image data with a medical imaging device, with said image data being used for precise radiation therapy. Likewise, within the framework of an operative intervention, it can be necessary to record image data with a medical imaging device in a sterile environment, wherein the operative intervention per se cannot take place in the position in which the image data was recorded. In these cases an immobile position of the patient is required, which still makes it possible to transport the patient, in particular to guarantee that the coordinate systems of the different medical devices match. Since the different medical devices typically comprise different patient tables or are compatible with different patient tables, patient positioning apparatuses embodied for mounting on different patient tables exist. The lower side of such a patient positioning apparatus is typically designed in such a way that it can be moved easily between different patient tables and/or other flat surfaces, such as a hospital bed for example, even if the patient is lying on the upper side of the patient positioning apparatus. Such a patient positioning apparatus can comprise an air cushion system, as described in WO2016115351A2 for example.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 illustrates a patient positioning apparatus according to an exemplary embodiment of the disclosure.

FIG. 2 illustrates a patient positioning apparatus to an exemplary embodiment of the disclosure.

FIG. 3 illustrates a patient positioning apparatus to an exemplary embodiment of the disclosure.

FIG. 4 illustrates a patient positioning apparatus to an exemplary embodiment of the disclosure.

FIG. 5 illustrates a medical imaging device to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure is to specify a patient positioning apparatus that makes possible an especially comfortable and/or safe medical procedure for the patient.

In an exemplary embodiment, the patient positioning apparatus comprises a compressor, a support surface having an upper side with at least one upper opening pore and a lower side with at least two lower opening pores, a valve having at least one first setting connecting the compressor to at least one lower opening pore of the at least two lower opening pores by means of a first line and a second setting connecting the compressor to the at least one upper opening pore by means of a second line, wherein the compressor is configured, in a basic setting as a compressor, to create an overpressure in the first line and/or the second line.

The support surface may be configured as a surface to support a patient from below. The support surface may be configured in such a way that a patient is able to be positioned lying down on its upper side. The support surface typically has a width of between 40 cm and 80 cm, preferably between 50 cm and 70 cm. The support surface typically has a length of between 170 cm and 230 cm, preferably between 190 cm and 210 cm. The support surface typically has a height of between 0.5 cm and 20 cm, preferably between 2 cm and 6 cm.

The compressor may be configured as a compressor and/or as a ventilator and/or as a vacuum pump. The compressor may be connected to the valve via an access line. The valve may be configured as a three-way valve and/or connects the access line to the first line in the first setting. The valve may be configured as a three-way valve and/or connects the access line to the second line in the second setting. The valve may be configured, in a third setting, to block the first line and the second line. As an alternative or in addition the valve can be configured to block the access line in the third setting. The valve may be able to be set in such a way that it has the first setting and/or the second setting and/or the third setting.

The first line and/or the second line and/or the access line are typically configured to channel a flow of air. The first line and/or the second line and/or the access line can each be configured as a pipe and/or hose and/or channel and/or be hollow.

The first line connects the valve to at least one lower opening pore. The compressor, the access line, the valve and the first line are preferably configured to create a flow of air in at least one lower opening pore. The at least two lower opening pores can be integrated into the lower side. A lower opening pore may be an opening in the lower side of the support surface. The lower opening pore can be integrated into the lower side of the support surface. The patient positioning apparatus comprises at least two, preferably at least four, especially preferably at least 10 lower opening pores. The lower opening pores are preferably arranged symmetrical to one another at least in part. The lower opening pores can be distributed evenly over the lower side.

The second line connects the valve to at least one upper opening pore. The compressor, the access line, the valve and the second line are preferably configured to create a flow of air in the at least one upper opening pore. The at least one upper opening pore can be integrated into the upper side. An upper opening pore may be an opening in the upper side of the support surface. The upper opening pore can be integrated into the upper side of the support surface. The patient positioning apparatus comprises at least one, preferably at least two, especially preferably at least four upper opening pores. The patient positioning apparatus preferably comprises at least eight, especially preferably at least ten upper opening pores.

If the patient positioning apparatus comprises a number of upper opening pores, then the number of upper opening pores are preferably arranged symmetrically to one another, at least in part. If the patient positioning apparatus comprises a number of upper opening pores, then the number of upper opening pores can be distributed evenly on the upper side.

If the patient positioning apparatus comprises a number of upper opening pores, then at least two thirds of the number of upper opening pores can be arranged on one half of the upper side.

The inventive patient positioning apparatus makes possible a controlled flow of air on the lower side and on the upper side of the support surface. In particular the patient is positioned on the upper side of the support surface during a medical examination of the patient, so that the patient positioning apparatus can control a flow of air over the patient. The flow of air on the upper side can be controlled with the same compressor as the flow of air on the lower side.

The flow of air on the upper side makes possible a controlled ventilation and/or an exchange of air in the area of the patient, whereby for example the number of viruses and/or bacteria in the area of the patient, in particular on the upper side, can be reduced. The flow of air can likewise be used for controlling the air in the room in the area of a patient positioned on the upper side. This makes especially comfortable and safe medical treatment, especially medical imaging, possible for the patient.

One form of embodiment of the patient positioning apparatus makes provision for the first line and/or the second line to be arranged within the patient positioning apparatus, in particular within the support surface. The first line and/or the second line are preferably enclosed in a housing of the support surface. This form of embodiment of the patient positioning apparatus, in particular of the support surface, is especially comfortable and safe of a patient supported on the upper side.

One form of embodiment of the patient positioning apparatus is configured to create an overpressure on the lower side of the support surface, in particular of an air cushion for the support surface, by using the compressor in the basic setting and by blocking the at least one upper opening pore by using the valve in the first setting. The valve in accordance with this form of embodiment is configured as a three-way valve and makes possible to direct a flow of air created in the compressor to at least one upper opening pore or to the at least two lower opening pores.

The patient positioning apparatus, in particular the combination of compressor, first line and the at least two lower opening pores, may be configured to create an overpressure on the lower side of the support surface, with said overpressure reducing the force of the weight of a patient positioned on the support surface at least at the positions of the at least two lower opening pores and/or at least partly compensating for it. This type of overpressure reduces the point of contact between the support surface and a surface positioned below it, such as a patient table for example, and thus acts as an air cushion between the support surface and a surface positioned below it. This makes it easier to relocate the patient in a lying position, for example between two patient tables, in particular when the relocation of the patient is inclusive of the patient positioning apparatus, in particular of the support surface. This is relevant in particular in the field of brachytherapy and cancer therapy or in the event of an evacuation of patients. This relieves the load on the medical personnel. The duration of the relocation is typically limited and a flow of air on the upper side can typically be dispensed with for this period. In particular a blocking of the upper opening pore makes possible an efficient creation of an especially high overpressure on the lower side of the support surface.

One form of embodiment of the patient positioning apparatus is configured to create an overpressure, in particular of a flow of air, on the upper side of the support surface by using the compressor in the basic setting and by blocking the at least two lower opening pores by using the valve in the second setting. The valve in accordance with this form of embodiment is configured as a three-way valve and makes it possible to direct a flow of air created in the compressor to at least one upper opening pore or to the at least two lower opening pores.

The patient positioning apparatus, in particular the combination of compressor, second line and the at least one upper opening pore, may be configured to create an overpressure on the upper side of the support surface, with said overpressure bringing about an air circulation and/or flow of air, for example with a speed of at most 10 km/h, preferably of at most 5 km/h. Such an overpressure makes possible a flow of air that is comfortable for a patient. The valve is typically used in the second setting typically during a medical procedure, in particular medical imaging, i.e. at a time at which typically no relocation of the patient is being undertaken and/or the patient is positioned as statically as possible and consequently flow of air and/or overpressure on the lower side is able to be dispensed with. This form of embodiment makes possible an efficient use of the compressor by the use of the valve in the second setting during a medical procedure, in particular medical imaging, and makes a convenient examination of the patient possible.

One form of embodiment of the patient positioning apparatus makes provision for the valve and/or the compressor to be configured in their basic setting in such a way that the overpressure at the upper opening pore is lower than the overpressure at the at least two lower opening pores. This in particular enables the overpressure at the at least two lower opening pores to be used for creating an air cushion between the support surface, in particular the lower side, and a further surface, whereas in a medical examination a comfortable flow of air over the patient is able to be set, with said flow of air supplying the patient with fresh air, preferably at the ideal temperature.

One form of embodiment of the patient positioning apparatus additionally comprises a warm air supply connected to the compressor and, by using the compressor in the basic setting and by using the valve in the second setting, is configured to create a warm flow of air on the upper side of the support surface.

The warm air supply can be integrated into the compressor and/or be comprised by the compressor. The warm air supply is typically configured to heat up ambient air. The compressor, together with the warm air supply, may be configured to create a warm flow of air, in particular in the access line. Preferably the valve has the second setting when the compressor is operated together with the warm air supply, in particular during a medical examination. Thus the temperature on the upper side can be especially well regulated and a patient positioned on the upper side getting cold can be avoided, even without the use of a cover. Dispensing with a cover is in particular hygienic, above all within the framework of an emergency procedure and/or an emergency examination. For pediatric patients in particular control of the body temperature and avoiding the patient getting too cold during an examination is essential, in particular with an examination that lasts a long time, such as for example magnetic resonance examinations.

One form of embodiment of the patient positioning apparatus makes provision for the compressor to be configured as a two-way compressor and to be configured, as an alternative to the basic setting, as a vacuum pump in its inverted setting to create a vacuum. The compressor in this form of embodiment is accordingly configured to invert a flow of air. In particular the compressor is configured in its inverted setting to create a flow of air from the at least one upper opening pore in the direction of the compressor, provided the valve has the second setting. The compressor in its inverted setting can be configured to create a flow of air from the at least two lower opening pores in the direction of the compressor, provided the valve has the first setting. This form of embodiment makes it possible to control the flow of air in the form a vent, whereby a regular exchange of air in the area of the patient and the medical personnel can be ensured especially well. This enables a medical examination to be done in a particularly sterile and safe manner and yet still at low cost. This is important in particular with medical imaging devices, which are used at least in part in the surgical environment or in the surgical and radiological environment, i.e. when the medical imaging device is not positioned in a clean room and/or sterile room. Preferably this form of embodiment makes it possible to dispense with separate, sterile enclosures for the medical imaging device and/or still makes it possible to reduce infections during surgical interventions that need checking by means of a medical imaging device during the surgical intervention. The cleaning effort can likewise be reduced.

One form of embodiment of the patient positioning apparatus is configured to create a vacuum on the upper side of the support surface, in particular of a flow of air in the direction of the at least one upper opening pore, by using the compressor in its inverted setting.

This form of embodiment preferably makes provision for blocking the at least two lower opening pores by using the valve in the second setting to create a vacuum on the upper side. The at least one upper opening pore preferably acts as a vent.

The patient positioning apparatus, in particular the combination of compressor, second line and the at least one upper opening pore, may be configured to create a vacuum on the upper side of the support surface, with said vacuum bringing about an air circulation and/or flow of air, for example with a speed of at most 10 km/h, preferably of at most 5 km/h. Such a vacuum enables a comfortable flow of air for a patient. The valve is typically used in the second setting during a medical procedure, in particular medical imaging, i.e. at a time during which typically no relocation of the patient is being undertaken and/or the patient is positioned as statically as possible and consequently a flow of air and/or overpressure on the lower side is able to be dispensed with. This form of embodiment makes possible an efficient use of the compressor for controlled venting of the air from the environment of the patient.

One form of embodiment of the patient positioning apparatus comprises a deactivator configured to deactivate at least one part of the at least one upper opening pore and/or at least one part of the at least two lower opening pores. The patient positioning apparatus in this form of embodiment typically comprises more than two, preferably more than four, especially preferably more than eight upper opening pores.

The deactivator can comprise switches and/or movable flaps, wherein a switch and/or movable flap may be configured for blocking and/or disabling, in particular for deactivating, an upper opening pore or lower opening pore. The deactivator preferably comprises a number of switches and/or movable flaps with said number corresponding to the number of the upper opening pores and/or z lower opening pores.

This form of embodiment makes possible an individual flow of air, which in particular is able to be adapted to the patient. Through the number of activated, i.e. non-deactivated, at least two lower opening pores the overpressure on the lower side can be controlled, in particular depending on a weight of a patient positioned on the support surface. This makes possible an especially good mobility of the support surface during relocation of the patient.

The number of activated, i.e. non-deactivated, upper opening pores enables the overpressure and/or vacuum on the upper side to be controlled, in particular depending on a size and/or shape of a patient positioned on the support surface. This makes possible an individual adaptation of the flow of air, whereby the exchange of air on the upper side can be ensured especially efficiently. In particular the exchange of air can be focused on specific regions of the body and thereby the patient's wishes in respect of temperature met.

One form of embodiment of the patient positioning apparatus comprises a sterile air supply arranged at a distance of at least 0.5 meters from the support surface and configured to create an at least partly sterile flow of air directed onto the upper side of the support surface.

The sterile air supply may be configured to emit sterile air. The sterile air supply is at a distance of at least 0.5 meters, preferably at least 1.0 meters, especially preferably at least 2 meters from the support surface, in particular from the upper side. The sterile air supply may be arranged above the support surface. The lower opening pores and/or the at least one upper opening pore are configured for venting the originally sterile air after it has flowed around the patient, provided the compressor is operated in the inverted setting. This form of embodiment makes possible a constant sterile flow of air in the area of the patient, i.e. in particular in the area of a surgical intervention. This protects the patient and the medical personnel against infections, since in particular aerosols are reduced.

A further inventive patient positioning apparatus comprises a compressor, a support surface having an upper side and a lower side with at least two lower opening pores, and a first line connecting the compressor with at least one lower opening pore of the at least two lower opening pores, wherein the compressor is configured as a two-way compressor and is configured in a basic setting as a compressor to create an overpressure in the first line and in an inverted setting as a vacuum pump to create a vacuum in the first line.

The further inventive patient positioning apparatus preferably comprises an upper side free from an upper opening pore. The lower opening pores are preferably configured as a vent when the compressor is operated in its inverted setting. This further inventive patient positioning apparatus is configured to create a vacuum on the lower side when the compressor is operated in its inverted setting or to create an overpressure when the compressor is operated in its basic setting. The overpressure may be created by operating the compressor in its basic setting during relocation of a patient lying on the support surface. The vacuum may be created by operating the compressor in its inverted setting during positioning of a patient with the support surface within a medical imaging device and/or during a medical procedure and/or medical imaging examination.

This further inventive patient positioning apparatus thereby makes possible a simple relocation of the patient and a safe and comfortable lying position of the patient through a controlled flow of air, preferably temperature-controlled, during an examination. The further inventive patient positioning apparatus in this case is very simple and thereby a very robust construction and is thus low-cost and can be employed in diverse ways.

One form of embodiment of the further inventive patient positioning apparatus makes provision for the first line to be arranged within the patient positioning apparatus, in particular within the support surface.

One form of embodiment of the further inventive patient positioning apparatus makes provision for the compressor to be configured in such a way that the vacuum at the at least one lower opening pore in the inverted setting is lower that the overpressure in the basic setting. This enables an air cushion to be created in the basic setting, whereas in the inverted setting a comfortable flow of air is able to be created for the patient.

The disclosure is furthermore based on a medical imaging device comprising a tubular patient receiving area and an inventive patient positioning apparatus or the further inventive patient positioning apparatus, configured for at least partial positioning within the patient receiving area. The medical imaging device can be configured for example as a magnetic resonance device and/or as a computed tomography device and/or as a PET and/or as an x-ray device. The medical imaging device has a tubular patient receiving area, i.e. preferably a round opening for receiving the patient. The depth of the tubular patient receiving area, in particular of the round opening, preferably amounts to between 10 cm and 250 cm. The depth of the tubular patient receiving area can be smaller than the diameter of the tubular patient receiving area. The depth of the tubular patient receiving area can be larger than the diameter of the tubular patient receiving area.

The patient positioning apparatus can be integrated into the medical imaging device. The patient positioning apparatus can also be installed separately from the medical imaging device. The patient positioning apparatus can be linked to the medical imaging device. The patient positioning apparatus may be able to be positioned in the horizontal direction at least partly within the tubular patient receiving area, in particular also when a patient is lying on the patient positioning apparatus.

One form of embodiment of the medical imaging device additionally comprises an air supply, wherein the compressor is configured as a two-way compressor and, in a basic setting, is configured as a compressor to create an overpressure in the first line and in its inverted setting is configured as a vacuum pump to create a vacuum in the first line.

The air supply is typically configured to create a flow of air on the upper side and/or in the direction of the upper side of the support surface, preferably comprising the air in the room in which the medical imaging device is arranged. The air supply can be designed as a ventilator. The air supply can be integrated into a housing surrounding the tubular patient receiving area. The air supply can be integrated into the tubular patient receiving area and/or be arranged on a housing surrounding the tubular patient receiving area. The compressor can be configured, in its inverted setting, to control the flow of air emanating from the air supply. This makes possible an individual temperature control of the upper side of the support surface and/or of the patient receiving area.

Further forms of embodiment of the inventive medical imaging device are configured in a similar way to the forms of embodiment of the inventive patient positioning apparatus. The advantages of the inventive medical imaging device essentially correspond to the advantages of the inventive patient positioning apparatus, which have been set out in detail above. Features, advantages or alternate forms of embodiment mentioned here can likewise be transferred to the other claimed subject matter and vice versa.

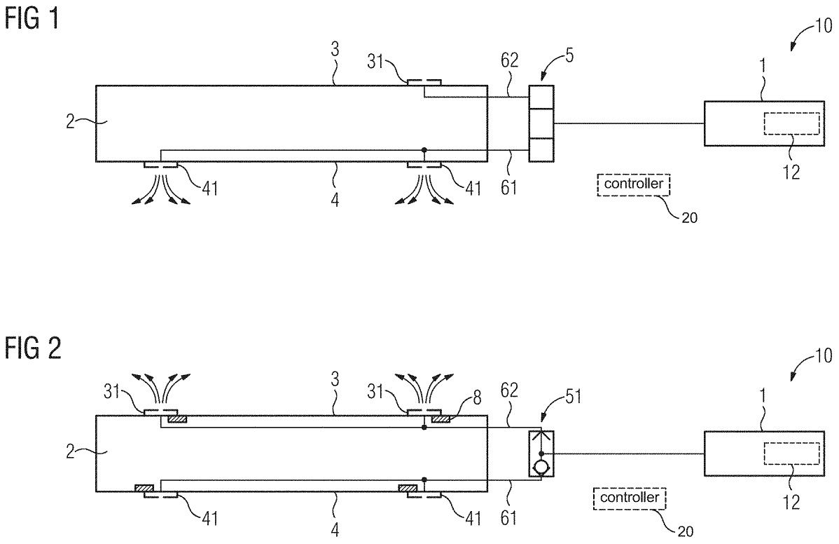

FIG. 1 shows an inventive patient positioning apparatus 10 in a schematic diagram of a first form of embodiment. The patient positioning apparatus 10 comprises a compressor 1, a support surface 2 having an upper side 3 and a lower side 4. The upper side 3 comprises an upper opening pore 31 and/or the one upper opening pore 31 is arranged on the upper side 3. The one upper opening pore 31 can also be integrated into the upper side 3. The upper side 3 can also comprise a number of upper opening pores 31. The lower side 4 comprises at least two lower opening pores 41 and/or the at least two lower opening pores 41 are arranged on the lower side 4. The at least two lower opening pores 41 can also be integrated into the lower side 4. The lower side 4 can also comprise more than two lower opening pores 41. The patient positioning apparatus 10 comprises a valve 5. The valve 5 is configured in such a way that, in a first setting, it connects the compressor 1 to at least one lower opening pore 41 of the at least two lower opening pores 41 by means of a first line 61. The valve 5 is configured in such a way that, in a second setting it connects the compressor 1 to the at least one upper opening pore 31 by means of the second line 62. The compressor 1 is configured, in a basic setting as a compressor, to create an overpressure in the first line 61 and/or the second line 62. The flow of air is shown that is created during operation of the compressor 1 with the valve 5 in the first setting: air flows out via the two lower opening pores 41. The first line 61 and/or the second line 62 are arranged within the support surface 2, i.e. between upper side 3 and lower side 4. The valve 5 and/or the compressor 1 are configured in the basic setting in such a way that the overpressure at the upper opening pore 31 is lower than the overpressure at the at least two lower opening pores 41. In an exemplary embodiment, the positioning apparatus 10 includes a controller 20 configured to control the operation of the valve 5, 51; the compressor 1, 11; deactivator 8; warm air supply 12; sterile air supply 7; and/or one or more other components of the positioning apparatus 10 and/or of the imaging device 17. Although not illustrated, the controller 20 may be communicatively coupled to the positioning apparatus 10 and/or one or more components thereof via one or more wired and/or wirelessly connections. Additionally or alternatively, one or more of the components of the positioning apparatus 10 may include a controller 20. In an exemplary embodiment, the controller 20 includes processing circuitry that is configured to perform one or more functions and/or operations of the controller 20. In an exemplary embodiment, the positioning apparatus 10 (and/or one or more components therein) additionally or alternatively includes one or more controllers configured to control the medical imaging device 17 (and/or one or more components of the imaging device 17).

The patient positioning apparatus 10 can optionally comprise a warm air supply 12, with said warm air supply 12 being connected to the compressor 1 and/or being integrated into the compressor 1. The warm air supply 12 is configured in combination with the use of the compressor 1 in the basic setting and of the valve 5 in the second setting to create a warm flow of air on the upper side 3 of the support surface 2.

FIG. 2 shows an inventive patient positioning apparatus 10 in a schematic diagram of a second form of embodiment. The second form of embodiment differs from the first form of embodiment by the valve, which is configured as changeover valve 51. The changeover valve 51 makes it possible to create an overpressure on the lower side 4 of the support surface 2 by using the compressor 1 in the basic setting and by blocking the at least one upper opening pore 31 by using the valve 5 in the first setting. The overpressure created in this way on the lower side 4 can serve as an air cushion between support surface 2 and a further surface, such as for example a patient table 16 Likewise the patient positioning apparatus 10 is configured through the changeover valve 51 to create an overpressure, in particular of a flow of air, on the upper side 3 of the support surface 2 by using the compressor 1 in the basic setting and by blocking of the at least two lower opening pores 41 by using the valve 5 in the second setting.

Regardless of the embodiment of the valve 5 as changeover valve 51, the second form of embodiment comprises a deactivator 8 configured to deactivate at least one part of the at least one upper opening pore 31 and/or at least one part of the at least two lower opening pores 41.

The deactivator 8 may be configured to activate and deactivate the lower opening pore 41 and upper opening pores 31 individually and/or separately in each case. The deactivation may be done by mechanical blocking. The diagram shows the flow of air that is created during operation of the compressor 1 with the valve 5 in the second setting. Air flows out via the two upper opening pores 31.

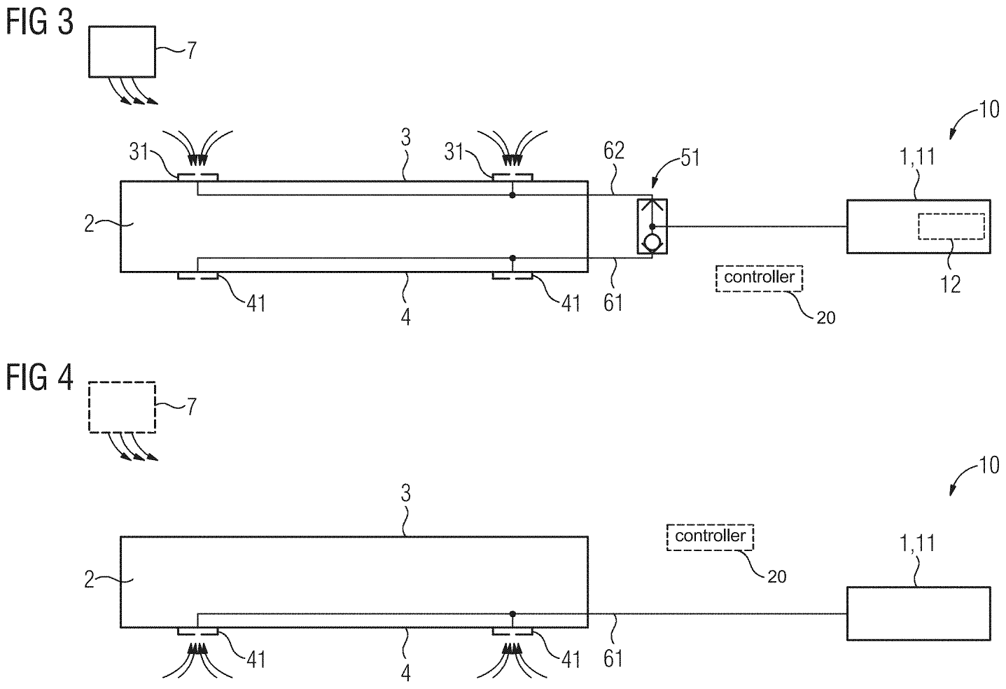

FIG. 3 shows an inventive patient positioning apparatus 10 in a schematic diagram of a third form of embodiment, in accordance with which the compressor 1 is configured as a two-way compressor 11 and, as an alternative to the basic setting, is configured in its inverted setting as a vacuum pump to create a vacuum. This compressor 1 in its inverted setting is configured in particular to create a vacuum on the upper side 3 of the support surface 2 and thus a flow of air in the direction of the at least one upper opening pore 31. Optionally the patient positioning apparatus 10 comprises a sterile air supply 7 arranged at a distance of at least 0.5 meters from the support surface 2, in particular from the upper side 3 of the support surface 2. The sterile air supply 7 may be configured to create an at least partly sterile flow of air directed onto the upper side 3 of the support surface 2.

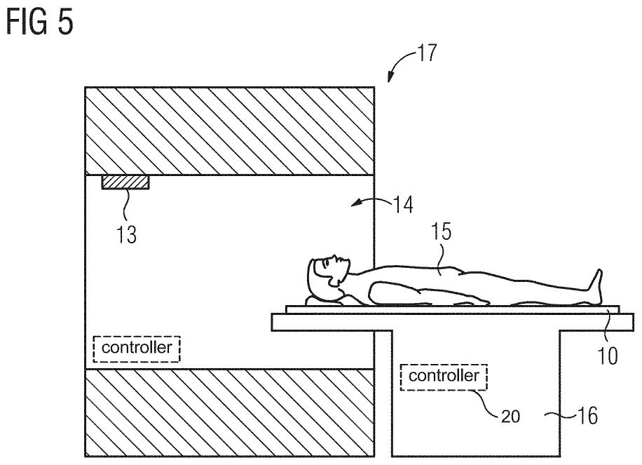

FIG. 4 shows a further inventive patient positioning apparatus 10 in a schematic diagram. This form of embodiment differs from the previous forms of embodiment in that the upper side 3 is free from upper opening pores 31 and the patient positioning apparatus 10 may be free from a valve 5. The first line 61 connects the compressor 1 to the two lower opening pores 41, wherein the compressor 1 is configured as a two-way compressor 11 and, in a basic setting, is configured as a compressor to create an overpressure in the first line 61 and in an inverted setting as a vacuum pump to create a vacuum in the first line 61.

Optionally the patient positioning apparatus 10 comprises a sterile air supply 7 arranged at a distance of at least 0.5 meters from the support surface 2. The sterile air supply 7 may be configured to create an at least partly sterile flow of air directed onto the support surface 2. The lower opening pores 41 are configured to receive the sterile flow of air created at least partly after it has flowed around the support surface 2, when the compressor 1 is operated in its inverted setting. In the basic setting the compressor 1 may be configured to create an overpressure on the lower side 4 of the support surface 2, which is able to be used as an air cushion for example.

FIG. 5 shows an inventive medical imaging device 17 in a schematic diagram. The medical imaging device 17 comprises a tubular patient receiving area 14 for receiving a patient 15, wherein the patient receiving area 14 is enclosed cylindrically in a circumferential direction by a housing of the medical imaging device 17. The patient 15 can be pushed by means of the patient table 16 of the medical imaging device 17 into the patient receiving area 14. The patient table 16 is arranged movably within the medical imaging device 17. The inventive patient positioning apparatus 10 is arranged on the upper side of the patient table 16 between the patient table 16 and the patient 15. The patient table 16 is thus configured, with the patient positioning apparatus 10, for at least partial positioning of the patient 15 within the patient receiving area 14. Optionally the medical imaging device 17 comprises an air supply 13.

Although the disclosure has been illustrated and described in greater detail by the exemplary embodiments, the disclosure is not restricted by the disclosed examples and other variations can be derived herefrom by the person skilled in the art, without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein. In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A patient positioning apparatus comprising:
a compressor;
a support surface having an upper side with at least one upper opening pore and a lower side with at least two lower opening pores; and
a valve configured to:
in a first setting, connect the compressor to at least one lower opening pore of the at least two lower opening pores by a first line; and
in a second setting, connect the compressor to the at least one upper opening pore by a second line,
wherein the compressor is configured to create an overpressure in the first line and/or the second line.

2. The patient positioning apparatus as claimed in claim 1, wherein the first line and/or the second line are arranged within the patient positioning apparatus.

3. The patient positioning apparatus as claimed in claim 1, wherein the first line and/or the second line are arranged within the support surface of the patient positioning apparatus.

4. The patient positioning apparatus as claimed in claim 1, wherein, in the first setting:
the valve is configured to block the at least one upper opening pore; and
the compressor is configured to create an overpressure on the lower side of the support surface to create an air cushion for the support surface.

5. The patient positioning apparatus as claimed in claim 1, wherein, in the second setting:
the valve is configured to block the at least two lower opening pores; and
the compressor is configured to create an overpressure on the upper side of the support surface to create a flow of air.

6. The patient positioning apparatus as claimed in claim 1, wherein the valve and/or the compressor are configured such that the overpressure at the upper opening pore is less than the overpressure at the at least two lower opening pores.

7. The patient positioning apparatus as claimed in claim 1, further comprising a warm air supply connected to the compressor, the warm air supply being configured to create a warm flow of air on the upper side of the support surface by using the compressor and by using the valve in the second setting.

8. The patient positioning apparatus as claimed in claim 1, wherein the compressor is a two-way compressor configured as a vacuum pump in an alternative setting to create a vacuum.

9. The patient positioning apparatus as claimed in claim 8, wherein the two-way compressor is configured to create a flow of air in a direction of the at least one upper opening pore to create the vacuum on the upper side of the support surface.

10. The patient positioning apparatus as claimed in claim 1, further comprising a deactivator configured to deactivate at least a portion of the at least one upper opening pore and/or at least a portion of the at least two lower opening pores.

11. The patient positioning apparatus as claimed in claim 1, further comprising a sterile air supply configured to create an at least partly sterile flow of air directed onto the upper side of the support surface.

12. The patient positioning apparatus as claimed in claim 11, wherein the sterile air supply is arranged at a distance of at least 0.5 meters from the support surface.

13. A patient positioning apparatus comprising:
a two-way compressor;
a support surface having an upper side and a lower side with at least two lower opening pores; and
a first line configured to connect the compressor to at least one lower opening pore of the at least two lower opening pores,
wherein the two-way compressor is configured to:
operate as a compressor in a first setting to create an overpressure in the first line, and
as a vacuum pump in a second setting to create a vacuum in the first line.

14. A medical imaging device comprising:
a tubular patient receiving area; and
a patient positioning apparatus configured to be at least partially positioned within the patient receiving area, the patient position apparatus including:
a compressor;
a support surface having an upper side with at least one upper opening pore and a lower side with at least two lower opening pores; and
a valve configured to:
in a first setting, connect the compressor to at least one lower opening pore of the at least two lower opening pores by a first line; and
in a second setting, connect the compressor to the at least one upper opening pore by a second line,
wherein the compressor is configured to create an overpressure in the first line and/or the second line.

15. The medical imaging device as claimed in claim 14, wherein the compressor is a two-way compressor that is configured to:
operate as a compressor in a first setting to create the overpressure in the first line and/or the second line, and
as a vacuum pump in a second setting to create a vacuum in the first line.

16. The medical imaging device as claimed in claim 14, further comprising an air supply, wherein the compressor is a two-way compressor configured to: operate as a compressor in a first setting to create an overpressure in the first line and, operate as a vacuum pump in a second setting to create a vacuum in the first line.
